(19) United States
(12) Patent Application Publication
(10) Pub. No.: US 2020/0337633 A1
(43) Pub. Date: Oct. 29, 2020

(54) SYSTEM AND METHOD FOR DETECTING AND/OR DETERMINING CHARACTERISTICS OF TISSUE

(71) Applicant: Briteseed LLC, Chicago, IL (US)

(72) Inventors: Amal Chaturvedi, Chicago, IL (US); Hariharan Subramanian, Mundelein, IL (US)

(21) Appl. No.: 16/962,177

(22) PCT Filed: Jan. 18, 2019
(86) PCT No.: PCT/US2019/014234

§ 371 Date: Jul. 14, 2020

Related U.S. Application Data
(60) Provisional application No. 62/618,924, filed on Jan. 18, 2018.

(51) Int. Cl. A61B 5/00; A61B 90/30; A61B 17/00; A61B 5/026

(52) U.S. Cl. CPC A61B 5/489; A61B 90/30; A61B 17/00; A61B 5/0261; A61B 2017/2926; A61B 2090/306; A61B 2017/00057; A61B 2017/00154; A61B 2505/05

(57) ABSTRACT

A surgical system used to detect tissue and/or determine tissue characteristics within a region proximate to a working end of a surgical instrument includes at least one light emitter disposed at the working end and configured to be activated to emit photons, and at least one light sensor disposed at the working end, facing in a common direction as the at least one light emitter, and configured to receive photons emitted from the at least one light emitter and exiting from the region, the at least one light sensor configured to receive photons over a limited time period. The system also includes a controller coupled to the at least one light sensor, the controller configured to operate the at least one light sensor to receive photons over the limited time period after a time delay from activation of the at least one light emitter.

### BACKGROUND

This patent is directed to a system and method for determining the characteristics of tissue, and in particular to a system and method using light exiting the same side of a surface as a light emitter.

Systems and methods that identify artifacts, and in particular vessels, in the surgical field during a surgical procedure provide valuable information to the surgeon or surgical team. In general terms, U.S. hospitals lose billions of dollars annually in unreimbursable costs because of inadvertent vascular damage during surgery. The involved patients face a mortality rate of up to 32%, and likely will require corrective procedures and remain in the hospital for an additional nine days, resulting in tens, if not hundreds, of thousands of dollars in added costs of care. Consequently, there is this significant value to be obtained from methods and systems that permit accurate determination of the presence of vessels, such as blood vessels, in the surgical field, such that these costs may be reduced or avoided.

Systems and methods that provide information regarding the presence of blood vessels in the surgical field are particularly important during minimally-invasive surgical procedures. Traditionally, surgeons have relied upon tactile sensation during surgical procedures both to identify blood vessels and to avoid inadvertent damage to these vessels. Because of the shift towards minimally-invasive procedures, including laparoscopic and robotic surgeries, surgeons have lost the ability to use direct visualization and the sense of touch to make determinations as to the presence of blood vessels in the surgical field. Consequently, surgeons must make the determination whether blood vessels are present in the surgical field based primarily on convention and experience. Unfortunately, anatomical irregularities frequently occur because of congenital anomalies, scaring from prior surgeries, and body habitus (e.g., obesity).

While the ability to determine the presence or absence of a vessel within the surgical field provides valuable advantages to the surgeon or surgical team and is of particular importance for minimally-invasive procedures where direct visualization and tactile methods of identification have been lost, the ability not simply to detect, but also to characterize, the identified vasculature provides additional important advantages. For example, it would be advantageous to provide information relating to the size of the vessel, such as the inner or outer diameter of the vessel. Size information is particular relevant as the Food and Drug Administration presently approves, for example, thermal ligature devices to seal and cut vessels within a given size range, typically less than 7 mm in diameter for most thermal ligature devices. If a thermal ligature device is used to seal a larger blood vessel, then the failure rate for a seal thus formed may be as high as 19%.

Further, it would be of assistance to be able to determine the type of tissue surrounding the vessel, not simply that the vessel is surrounded by tissue. Characterization of the non-vascular tissue, such as its depth overlying a detected vessel, would provide still further advantages.

In addition, it would be preferable to provide this information with minimal delay between vessel or tissue detection and analysis, such that the information may be characterized as real-time or near real-time (e.g., <2 seconds). If considerable time is required for analysis, then at a minimum this delay will increase the time required to perform the procedure. In addition, the delay may increase surgeon fatigue, because the surgeon will be required to move at a deliberate pace to compensate for the delay between motion of the instrument and delivery of the information. Such delays may in fact hinder adoption of the system, even if the information provided reduces the risk of vascular injury.

Further, it would be advantageous to detect and analyze the vasculature and other tissues without the need to use a contrast medium or agent. While the use of a contrast agent to identify vasculature has become conventional, the use of the agent still adds to the complexity of the procedure. The use of the agent may require additional equipment that would not otherwise be required, and increase the medical waste generated by the procedure. Further, the use of the contrast agent adds a risk of adverse reaction by the patient.

As set forth in more detail below, the present disclosure describes a surgical system including a system and method for detecting tissue and/or determining tissue characteristics, such as vessel presence, vessel size, tissue type, and tissue depth, embodying advantageous alternatives to the existing methods, which may provide for improved identification for avoidance or isolation of tissues.

### SUMMARY

According to an aspect of the present disclosure, a surgical system used to detect tissue and/or determine tissue characteristics within a region proximate to a working end of a surgical instrument includes at least one light emitter disposed at the working end of the surgical instrument and configured to be activated to emit photons, and at least one light sensor disposed at the working end of the surgical instrument, facing in a common direction as the at least one light emitter, and configured to receive photons emitted from the at least one light emitter and exiting from the region, the at least one light sensor configured to receive photons over a limited time period. The system also includes a controller coupled to the at least one light sensor, the controller configured to operate the at least one light sensor to receive photons over the limited time period after a time delay from activation of the at least one light emitter.

According to another aspect of the present disclosure, a method of detecting tissue and/or determining tissue characteristics within a region proximate to a working end of a surgical instrument includes emitting photons at the working end of the surgical instrument in the direction of a surface of the region, sensing photons exiting from the surface at the working end of the surgical instrument over a limited time period delayed from the emitting of the photons, generating a signal based on the photons sensed at the working end of the surgical instrument, and detecting tissue and/or determining tissue characteristics within the region proximate to the working end of the surgical instrument based on the signal.

### BRIEF DESCRIPTION OF THE DRAWINGS

The disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings is necessarily to scale.

FIG. 1 is a schematic diagram of a surgical system according to an embodiment of the present disclosure;

FIG. 2 is a schematic diagram of a surgical system according to another embodiment of the present disclosure;

FIG. 3 is an enlarged, fragmentary view of an embodiment of the surgical instrument of FIG. 1 with light emitter and light sensor with fixed spacing, and a section of a vessel illustrated as proximate the light emitter and light sensor;

FIG. 4 is an enlarged, fragmentary view of an embodiment of the surgical instrument of FIG. 2 with light emitter and light sensor with fixed spacing, and a section of an vessel illustrated as proximate the light emitter and light sensors;

FIG. 5 is a schematic diagram of a light emitter/light sensor system according to an embodiment of the present disclosure;

FIG. 6 is a method of operating the surgical system according to an embodiment of the present disclosure;

FIG. 7 is a schematic diagram of a surgical system according to an embodiment of the present disclosure, in combination with an embodiment of a video system, illustrating the surgical system in use with the video system;

FIG. 8 is a schematic diagram of a surgical system according to an embodiment of the present disclosure, in combination with another embodiment of a video system, illustrating the surgical system in use with the video system;

FIG. 9 is a chart illustrating the ratio of photons counted using a 1 cm thick block or a 3 cm thick block taken relative to the photons counted using a 1 cm thick block, relative to the delay of the operation of the light sensor (SPAD detector); and

FIG. 10 is a chart illustrated the percentage of photons collected from a 3 cm sample relative to the photons counted using a 1 cm thick sample, relative to the time delay of the operation of the light sensor (SPAD detector).

### DETAILED DESCRIPTION OF VARIOUS EMBODIMENTS

A surgical system according to an embodiment of the present disclosure includes at least one light emitter, at least one light sensor, and a controller. The system may also include a surgical instrument as well.

The system may be used to detect tissue, e.g., determine the presence of a vessel within a region proximate to a working end of the surgical instrument. In particular, it is believed that the system may be used to determine the presence of a vessel within the region proximate to the working end of the surgical instrument regardless of the presence or the type of tissue surrounding the vessel. The embodiments of the system described below perform determinations relative to the presence of the vessel within the targeted region based on the light exiting the region (which may be referred to as back-scattered photons) as determined by the light sensor. According to other embodiments, it may be possible to determine other characteristics of the tissue, e.g., vessel, such as its depth, or to determine if other types of tissue (other than vessels) are present and to differentiate between the different tissue types.

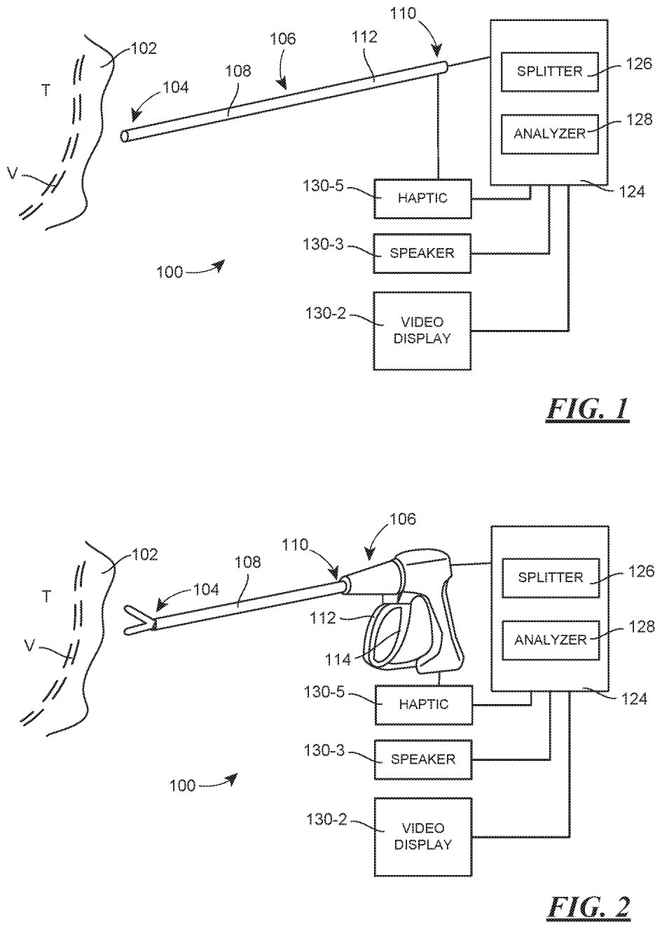

FIGS. 1 and 2 illustrate an embodiment of such a surgical system 100 used to determine the presence and/or other characteristics of a vessel, V, disposed within a region 102 of tissue, T, proximate to a working end 104 of a surgical instrument 106. It will be understood that the vessel V may be connected to other vessels with the region 102 of tissue T, and in addition, the vessel V may extend beyond the region 102 so as to be in fluid communication with other organs (e.g., the heart) also found in the body of the patient. Furthermore, while the tissue T appears in FIGS. 1 and 2 to surround fully the vessel V (in terms of both circumference and length) to a particular depth, this need not be the case in all instances where the system 100 is used. For example, the tissue T may only partially surround the circumference of and/or only surround a section of the length of the vessel V, or the tissue T may overlie the vessel V in a very thin layer. As further non-limiting examples, the vessel V may be a blood vessel, and the tissue T may be connective tissue, adipose tissue and/or liver tissue.

According to the illustrated embodiments, the working end 104 of the surgical instrument 106 is also a distal end of a shaft 108. Consequently, the working end and the distal end will be referred to as working end 104 or distal end 104. The shaft 108 also has a proximal end 110, and a grip or handle 112 (referred to herein interchangeably as grip 112) is disposed at the proximal end 110 of the shaft 108. The grip 112 is designed in accordance with the nature of the instrument 106; as to the dissector illustrated in FIG. 1, the grip 112 may be defined along a length of the shaft 108, while as to the thermal ligation device illustrated in FIG. 2, the grip 112 may be a pistol-type grip including a trigger 114. As a further alternative, finger rings arranged in a generally scissors-type grip may be used.

While the working or distal end 104 and the proximal end 110 with grip 112 are illustrated as disposed at opposite-most ends of the shaft 108, it will be recognized that certain surgical instruments have working ends (where a tool tip is attached, for example) disposed on the opposite-most ends of the shaft and a gripping region disposed intermediate to the opposite working ends. In accordance with the terms “distal” and “proximal” as used herein, the working ends of such an instrument are referred to herein as the distal ends and the gripping region as the proximal end. Relative to the illustrated embodiments, however, the distal and proximal ends are located at opposite-most (or simply opposite) ends of the shaft 108.

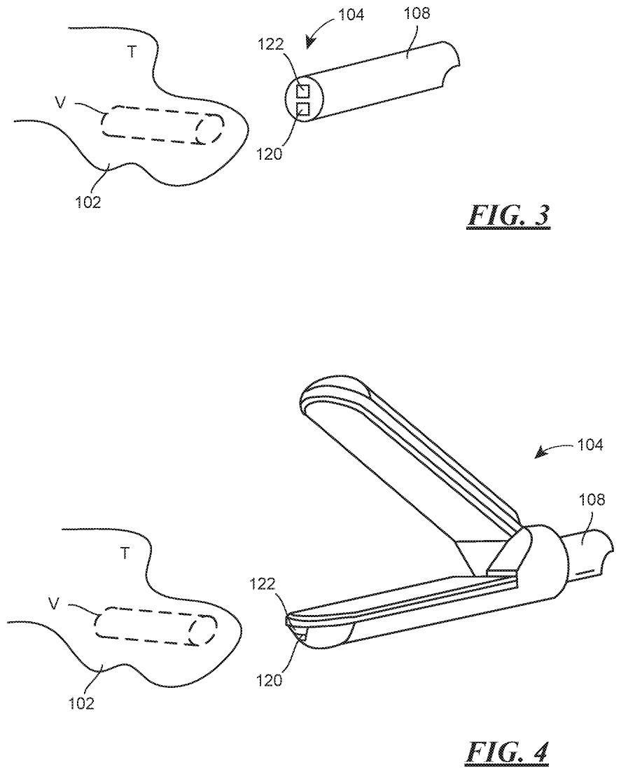

As mentioned above, according to the preferred embodiments illustrated, the surgical system 100 includes a sensor with at least one light emitter 120 (or simply the light emitter 120) and at least one light sensor or detector 122 (or simply the light sensor 122). See FIGS. 3 and 4. According to the illustrated embodiments, a controller 124 is coupled to the light emitter 120 and the light sensor 122, which controller 124 may include a splitter 126 and an analyzer 128 as explained below. See FIGS. 1 and 2.

The light emitter 120 and the light sensor may both be disposed at the working end 104 of the surgical instrument 106. While the emitter 120 and the sensor 122 are described as disposed at the working end 104 of the surgical instrument 106, it will be recognized that not all of the components that define the emitter 120 and the sensor 122 need be disposed at the working end of the instrument 106. The emitter 120 may include a length of optical fiber (e.g., a single mode optical fiber) and a light source (e.g. a laser), and the light source may be disposed remotely from the working end 104 and the fiber may have a first end optically coupled to the source and a second end disposed at the working end 104. According to the present disclosure, such an emitter 120 would still be described as disposed at the working end 104 because the light is emitted in the direction of the tissue at the working end 104 of the instrument 106. A similar arrangement may be described for the sensor 122 wherein an optical fiber (e.g., a multimode optical fiber) has a first end disposed facing the tissue and a second end optically coupled to other components that collectively define the sensor 122.

As illustrated in FIGS. 3 and 4, the system 100 may be configured with the light emitter 120 and the light sensor 122 facing in a common general direction (in the alternative to facing each other or being opposite each other), for example on a blunt end of a laparoscopic tool or dissector (e.g., a Kittner dissector or suction irrigator—FIG. 3) or on a single jaw of a two-jaw device, such as a thermal ligation device (FIG. 4), although the relative angle between the light emitter 120 and light sensor 122 may be fixed or variable.

As illustrated in FIGS. 1-4, the light emitter 120 and light sensor 122 are disposed generally facing in a common direction (i.e., the direction of the tissue sample of interest). This does not require the emitter 120 and the sensor 122 to be generally disposed in a common plane, although this is preferred. According to certain embodiments, the emitter 120 and sensor 122 may be formed integrally (i.e., as one piece) with one of the jaws of a surgical instrument 106 (see FIGS. 2 and 4), although other options are possible, as discussed above. In this manner, light emitted by the emitter 120 and scattered by the tissue of interest may be captured by the light sensor 122.

According to one embodiment, the light emitter 120 may be a pulsed laser, for example, having a pulse width of 50 ps and a wavelength of 640 nm. As one non-limiting example, the pulsed laser may be the PDL800D model, manufactured by Picoquant of West Springfield, Mass. The light sensor 122 may be a time-gated single photon avalanche diode (SPAD) detector with an active area of 100 μm. As one non-limiting example, the SPAD detector may be manufactured by Micro Photon Devices of Bolzano, Italy.

FIG. 5 illustrates in detail the equipment that may be part of such an embodiment of the system 100. In particular, as mentioned above, the light emitter 120 may include a laser 140 and a first optical fiber 142 having a first end 144 coupled to the laser 140 and a second end 146 disposed at the working end 104. The light sensor 122 may include the SPAD detector 148 and a second optical fiber 150 having a first end 152 coupled to the SPAD detector 148 and a second end 154 disposed at the working end 104. The light sensor 122 may include a photon counting module 156 that is coupled to the SPAD detector 148. The laser 140 may be coupled to the photon counting module (or counter) 156 via a delay circuit 158 (which may be in the form of a software-implemented delay instead of an electronic circuit) to implement the delay between activation of the emitter 120 and the operation of the sensor 122. The photon counting module 156 is also coupled to a time correlated single photon counting module (or counter) 160 that can provide a real-time photon count.

The use of the pulsed laser light emitter 120 and the SPAD detector sensor 122 may provide certain advantages over other possible emitter/sensor pairings with the system 100 operated in accordance with embodiments of the present disclosure.

By way of explanation and as noted in previous applications, it has been our belief that the spacing between a light emitter and a light sensor can influence the light received by the sensor where the emitter and sensor are configured to be used facing the surface of a region of interest. As presently understood, after photons leave the emitter and contact the tissue, an ensemble of independent photons will return to the same surface. Some of the detected photons travel a short distance from the plane of the emitter and detector and exit, while other photons travel farther into the tissue (up to distances exceeding 1 cm) before exiting at the surface without being absorbed (photons that are absorbed cannot contribute to the photocurrent). Path length distributions and the penetration depth of photons that reach the sensor vary as a function of emitter-sensor separation, with maximum effective photon depth penetration values several times greater than the physical emitter-sensor separation.

It is further believed that adjusting the angle of the emitter 120 and/or sensor 122 may provide a similar effect. That is, similar to the way in which a change in the linear distance between the emitter 120 and the sensor 122 allows for the sampling of a different proportion of long-traveling photons at the surface sensor 122, a variation in angle of the emitter 120 and/or sensor 122 can change the depth and the distance to which the photons travel before being sampled by the sensor 122. As a consequence, changes in the angle of the emitter and/or sensor are believed to permit the depth at which vessels can be detected by the instrument 106 to be varied.

While we have suggested the use of fixed or variable spacings or angles between the light emitter and light sensor to permit determination of tissue characteristics at different depths, there is a drawback to such systems, in particular to systems that use wider spacings between the light emitter and light sensor to determine the characteristics of tissue at greater depths. Minimally invasive surgical tools are sized to fit within small incisions in the body, and thus space is at a premium. For example, Kittner or blunt-tipped dissectors may have a diameter that is approximately 5 mm. As such, the spacing between the light emitter and light sensor possible in such a system may not be able to determine tissue characteristics deeper than 2 mm into the tissue, for example.

In the alternative to the use of different spacings to determine tissue characteristics at different depths, a time-delayed, time-gated sensor approach is proposed herein. It is believed that the time-delayed, time-gated sensor approach will be useful to determine tissue characteristics at deeper depths, even though the spacing between light emitter and light sensor is relatively small.

Specifically, it is presently understood that photons from different depths exit the surface of the tissue at different positions on the surface. In fact, the number of photons coming from deep regions may be higher at smaller distances from the emitter (e.g., less than 5 mm) than at larger distances from the emitter. Unfortunately, it is also recognized that the number of photons coming from shallow regions (less than 0.2 cm) is also higher at these smaller distances from the emitter. In fact, the number of photons coming from shallow regions at the smaller distances may be 95% of all photons detected. As such, the number of photons from shallower depths make it difficult (if not impossible) to differentiate the photons coming from the deeper depths and exiting at the small distances from the emitter. Consequently, the detection is performed at larger distances from the emitter where the number of photons returning from the deeper depths is a larger percentage of the overall number of returning photons.

It is believed that at the smaller distances from the emitter, where the number of photons returning to the surface is larger for both shallow depths and deeper depths, the photons returning to the surface from shallower depths do so more quickly than the photons returning to the surface from deeper depths. As a consequence, it is believed that a closer spacing between light emitter and light sensor may be permissible if the sensor can be operated to ignore the photons returning from the shallower depths and to capture the photons returning from the deeper depths as a matter of timing.

To this end, the light sensor 122 is controlled by the controller 124 to operate after a time delay from the activation of the light emitter 120. For example, the light sensor 122 may be maintained in an “off” state while the light emitter 120 is shined on the tissue, and then the light sensor 122 may be operated in an “on” state to capture the photons arriving from the deeper depths. A time delay between the activation of the light emitter 120 and the operation of the light sensor 122 on the order of several picoseconds may be all that is required to permit the capture of photons coming from the deeper depths of the tissue, and thus to permit determination of the tissue characteristics at these deeper depths.

It is not necessary for the time delay to be a constant value (or fixed) for all embodiments. According to some embodiments, the time delay may be varied according to the purpose to which the system 100 is being used. For example, if it is desired to determine the depth of the tissue or the characteristics of the tissue at different depths, the time delay may be varied to capture photons exiting the tissue from different depths. Varying the time delay also may be used to detect tissue type at different depths, or even to provide a size estimation of a blood vessel.

While the time delay between the activation of the light emitter 120 and the operation of the light sensor 122 may be approximately several picoseconds, it is believed that the duration of operation of the light sensor 122 will be relatively longer. That is, it is presently believed that the exposure time of the SPAD detector, or gate width, should be larger than 1 ns, because it is believed that 1 ns (or smaller) is insufficient time to collect meaningful information. On the other hand, a gate width in excess of 10 ns may be too large, causing degradation of the signal-to-noise ratio because of unwanted photons being captured by the light sensor 122. One factor in setting a proper gate width may be the desired depth resolution.

According to one embodiment of a method for operating the system 100 as explained above, the light emitter 120 (including laser 140) may be activated, and then the light sensor 122 (including SPAD detector 158) may be operated with a 0.5 ns delay. According to this embodiment, the activation of the light emitter 120 and the operation of the light sensor 122 will be repeated every 12.5 ns. This repeated cycling between activation of the light emitter 120 and operation of the light sensor 122 may occur for one second, for example, to create a temporal signal.

A temporal signal provides certain opportunities. If the photons from the light emitter 120 pass through non-vascular tissue, it would be expected that the number of photons returning to the sensor 122 will be approximately constant over time. On the other hand, if the photons from the light emitter 120 encounter a blood vessel in their path, it is believed more photons will be absorbed when the blood flows through the blood vessel than when it does not. Because blood flows through a blood vessel in a time-varying fashion, the increased absorption of the photons (when it occurs) will cause the number photons returning to the light sensor 122 to vary. As such, a temporal signal created by an embodiment of the system 100 may have pulsatile nature or component when a vessel, such as a blood vessel, is present.

In fact, the individual light sensor 122 may generate a signal comprising a first pulsatile component and a second non-pulsatile component. It will be recognized that the first pulsatile component may be an alternating current (AC) component of the signal, while the second non-pulsatile component may be a direct current (DC) component. While the AC waveform may correspond to the light affected by the pulsatile blood flow within the vessel, the DC component may correspond principally to light scattered by the superficial tissues.

Thus, according to the disclosed embodiments, the controller 124 may include a splitter 126 to separate the first pulsatile component from the second non-pulsatile component for the light sensor 122. The controller 124 also includes an analyzer 128 to determine at least the presence the vessel V within the region 102 proximate to the working end 104 of the surgical instrument 106 based on the pulsatile component. To display, indicate or otherwise convey the presence of the vessel V within the region 102, the controller 124 may be coupled to an output device or indicator 130 (see FIG. 1), which may provide a visible, audible, tactile or other signal to the user of the instrument 106.

According to certain embodiments, the splitter 126 and the analyzer 128 may be defined by one or more electrical circuit components. According to other embodiments, one or more processors (or simply, the processor) may be programmed to perform the actions of the splitter 126 and the analyzer 128. According to still further embodiments, the splitter 126 and the analyzer 128 may be defined in part by electrical circuit components and in part by a processor programmed to perform the actions of the splitter 126 and the analyzer 128.

For example, the splitter 126 may include or be defined by the processor programmed to separate the first pulsatile component from the second non-pulsatile component. Further, the analyzer 128 may include or be defined by the processor programmed to determine the presence of (or to quantify the size of) the vessel V within the region 102 proximate to the working end 104 of the surgical instrument 106 based on the first pulsatile component. The instructions by which the processor is programmed may be stored on a memory associated with the processor, which memory may include one or more tangible non-transitory computer readable memories, having computer executable instructions stored thereon, which when executed by the processor, may cause the one or more processors to carry out one or more actions.

For example, it is believed that a pulsatile signal will have a higher variance than a non-pulsatile one that can be quantified by using the standard deviation of the signal, defined herein as a Variance Metric (VM). Additionally, the percentage difference between the maximum and minimum eigenvalues, defined as Eigen Metric (EM), will be high (>60%) for a periodic signal and low (<60%) for a constant signal. A combination of EM and VM may provide a robust pulsatile signal detection mechanism that in turn may be used to determine tissue characteristics, such as the detection of a blood vessel and/or its other characteristics (e.g., diameter) or the depth of the surrounding tissue.

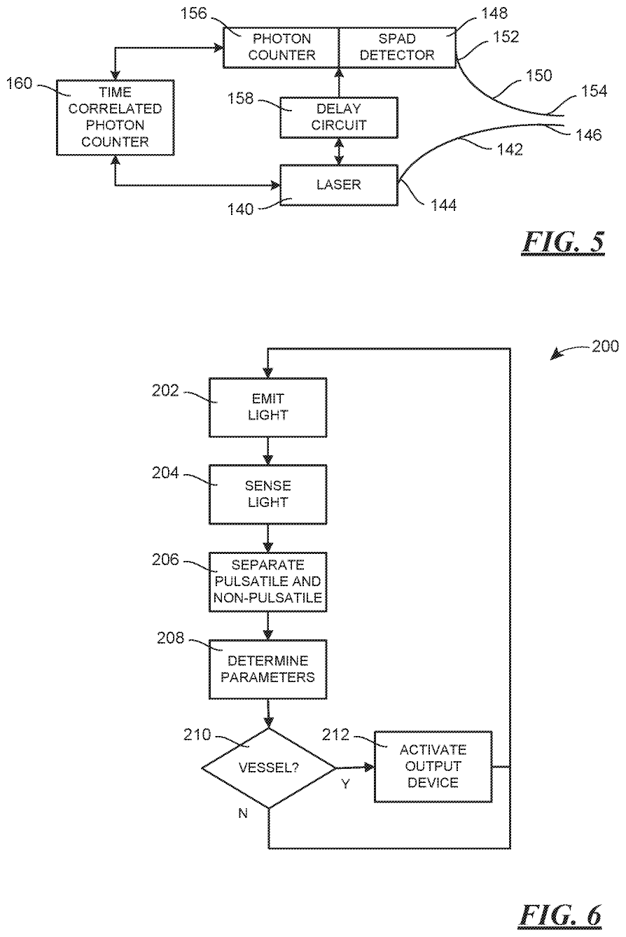

As such, a method 200 of determining if the presence of a vessel V within a region 102 proximate to a working end 104 of a surgical instrument 106 may be described. The method 200 may be carried out, for example, using a system 100 as described above in regard to FIG. 1. As illustrated in FIG. 6, the method 200 of operating the system 100 includes emitting photons (from a pulsed laser, for example) at a working end 104 of a surgical instrument 106 in the direction of a region at block 202, and sensing photons exiting from the region at the working end 104 of the surgical instrument 106 over a limited time period delayed from the emission of the photons at block 204. The method 200 continues at block 206 wherein a pulsatile component is separated from a non-pulsatile component of the signal generated by the light sensor. At block 208, one or more parameters are determined based on the pulsatile component of the signal, as suggested above.

At block 210, the parameters are interrogated to determine characteristics of a tissue proximate to the emitter 120/sensor 122 pair. According to one embodiment, the interrogation may simply be whether a vessel is present proximate the working end 104 of the instrument 106. If there is a vessel present, the method 200 may proceed to block 212, and activate one or more of the output devices 130 (e.g., a “vessel present” message displayed on the display 130-2, for example). If no vessel is present, then according to this embodiment, no output device 130 is activated, although it will be recognized that an alternative output device could instead be activated or the output device 130 activated in block 212 could be activated, but a different indication provided to the user (e.g., a “no vessel” message displayed on the display 130-2, for example).

The specific method for carrying out the interrogation may vary, but one embodiment includes calculating one or more parameters, and comparing each of the parameters to one or more thresholds. The thresholds may be set using empirically derived data, for example, or theoretically determined. Typically, but not necessarily, the comparison includes determining if the parameter exceeds a predetermined threshold. If a certain number of comparisons suggest that a vessel is present, then the method indicates that a vessel is present proximate to the working end 104 of the instrument 106. Alternatively, the method provides no indication to the operator or user.

More particularly, the method may use a variable, or count, to store information regarding the number of comparisons that suggest a vessel is present. Each time the method determines that one of the comparisons suggests that a vessel is present, the count is increased by one. If the comparison does not suggest that a vessel is present, then the count is not increased. In a final step, the count is compared to a further threshold, defined in accordance with the aforementioned criteria (i.e., two or more favorable comparisons indicating a vessel is present, less than two indicating only tissue).

It will be recognized that the general operation of the method may be varied in a number of ways. For example, a greater or lesser number of parameters may be included in the determination. In addition, the sensitivity of the comparison need not be an all-or-nothing comparison, but a range of values may be assigned based on the comparison of the calculated parameter and preexisting empirically or theoretically determined thresholds (or ranges). Also, the use of a single variable to store the results of each comparison may be replaced by a variety of different options, such as the setting of a flag (e.g., 1/0 or T/F) for each of the comparisons, which flags are then read once all of the comparisons have been made. Other embodiments may implement further alternatives in addition to or in substitution for these enumerated options.

While the pulsatile, or AC, component may be used to determine if the vessel is present or not, the DC profile may be used to adapt the intensity emitted by the light emitter 120. In particular, it is believed that the intensity of the light emitter 120 plays an important role in the accuracy of vessel detection (and potentially tissue type and/or vessel size determination). If the intensity of the light emitter 120 is set too low, few photons may return from the deeper depths of the tissue. In such a circumstance, the sensor 122 may not be able to detect the pulsatile nature of the vessel, and it may be difficult to differentiate the vessel (e.g., artery) from the surrounding tissue (i.e., low resolution). Similar error may result if the intensity is set too high—too many photons may return and saturate the SPAD detector. Therefore, it would be desirable to provide a method and mechanism for selection of the intensity of the light emitter 120 that would limit the consequences of using an intensity that was either too low or too high for conditions.

For example, the amplitude of the DC component may be compared to a predetermined value, or range, and if the calculated amplitude is equal to the value or within the range, the intensity is unchanged. To the extent that the amplitude is not equal to the value or falls outside the range, then the intensity is changed, with the increase or decrease in intensity dependent upon on whether the amplitude is greater than the upper limit of the range or below the lower limit of the range, for example. According to one embodiment, the range may be empirically derived.

While the general structure and operation of the system 100 has been described above, additional equipment of the system is discussed in detail.

For example, the light sensor 122 may include a mechanism for physically excluding photons reaching the sensor 122 from a range of angles. This mechanism can consist of a mask or grated layer to physically filter any photons that are not reaching the sensor 122 at a nearly perpendicular angle. It has been observed that the mean depth penetration of the photons leaving the emitter 120 is equal to just over half the distance of source-detector separation (˜2.5 mm penetration for our 5 mm spacing). This mechanism will increase the proportion of long-traveling and deep penetrating photons that are received by the sensor 122 thus increasing the depth at which the vessels can be detected by the instrument.

As for the indicator 130 used in conjunction with controller 124, a variety of output devices may be used. As illustrated in FIG. 1, a light emitting diode may be attached to or incorporated into the associated surgical instrument 106, and may even be disposed at the working end 104 of the instrument 106. Alternatively or in addition, an alert may be displayed on a video monitor 130-2 being used for the surgery, or may cause an image on the monitor to change color or to flash, change size or otherwise change appearance. The indicator 130 may be in the form of or include a speaker 130-3 that provides an auditory alarm. The indicator 130 also may be in the form of or may incorporate a safety lockout associated with the surgical instrument 106 that interrupts use of the instrument 106. For example, the lockout could prevent ligation or cauterization where the surgical instrument 106 is a thermal ligature device. As a still further example, the indicator 130 also may be in the form of a haptic feedback system, such as a vibrator 130-5, which may be attached to or formed integral with a handle or handpiece of the surgical instrument 106 to provide a tactile indication or alarm. Various combinations of these particular forms of the indicator 130 may also be used.

As mentioned above, the surgical system 100 may also include the surgical instrument 106 with the working end 104, to which the light emitter 120 and light sensor 122 are attached (in the alternative, removably/reversibly or permanently/irreversibly). The light emitter 120 and the light sensor 122 may instead be formed integrally (i.e., as one piece) with the surgical instrument 106. It is further possible that the light emitter 120 and light sensor 122 be attached to a separate instrument or tool that is used in conjunction with the surgical instrument or tool 106, such as a blunt end of a dissection tool.

As noted above, the surgical instrument 106 may be a thermal ligature device in one embodiment. In another embodiment, the surgical instrument 106 may simply be a grasper or grasping forceps having opposing jaws. According to still further embodiments, the surgical instrument may be other surgical instruments such as surgical staplers, clip appliers, and robotic surgical systems, for example. According to still other embodiments, the surgical instrument may have no other function that to carry the light emitters/light sensors and to place them within a surgical field. The illustration of a single embodiment is not intended to preclude the use of the system 100 with other surgical instruments or tools 106.

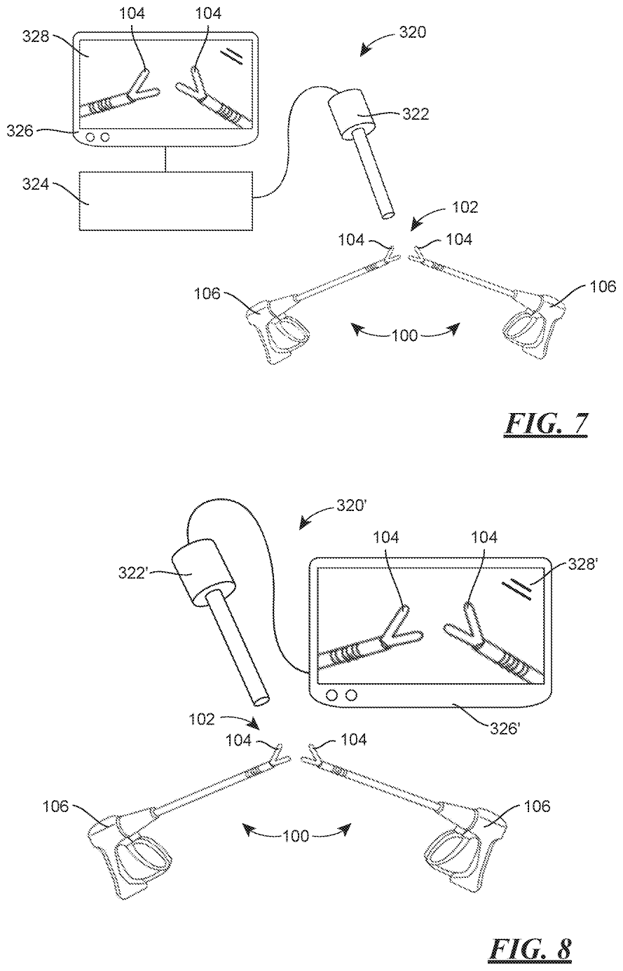

FIGS. 7 and 8 illustrate embodiments of the surgical system 100 in combination with embodiments of a video system 320, such as may be used conventionally during minimally invasive surgery or laparoscopic surgery, for example.

In the embodiment of FIG. 7, the video system 320 includes a video camera or other image capture device 322, a video or other associated processor 324, and a display 326 having a viewing screen 328. As illustrated, the video camera 322 is directed at the region 102 proximate the working ends 104 of two surgical instruments 106. As illustrated, both of the surgical instruments 106 are part of an embodiment of a surgical system 100. The other elements of the surgical system 100 are omitted for ease of illustration, although it will be noted that elements of the system 100, such as the splitter 126 and the analyzer 128, may be housed in the same physical housing as the video processor 324. The signal from the video camera 322 is passed to the display 326 via the video processor 324, so that the surgeon or other member of the surgical team may view the region 102 as well as the working ends 104 of the surgical instruments 106, which are typically inside the patient.

FIG. 8 illustrates another embodiment of a video system 320 that can be used in conjunction with an embodiment of the surgical system 100. According to this embodiment, the video processor 324 is not disposed in a housing separate from the video camera 322′, but is disposed in the same housing as the video camera 322′. According to a further embodiment, the video processor 324 may be disposed instead in the same housing as the remainder of the display 326′ as the display screen 328′. Otherwise, the discussion above relative to the embodiment of the video system 320 illustrated in FIG. 7 applies equally to the embodiment of the video system 320 illustrated in FIG. 8.

It will be understood that other aspects of the system 100 illustrated in FIGS. 1 and 2 could be incorporated into the system 320 illustrated in FIGS. 7 and 8. For example, the indicator 130-2 may refer to the display 326, 326′, and the other indicators described with reference to FIG. 1 (e.g., speaker 130-3 or haptic feedback 130-5) may be incorporated into the system 320.

### EXAMPLES

Experiments have been conducted using an embodiment of the above-described system. The experiments and results are reported below.

A first set of experiments were performed with a set of silicone phantom blocks of different thicknesses (1 cm and 3 cm). The system used was as generally described above, except that optical fibers were not used in conjunction with the laser or the SPAD detector.

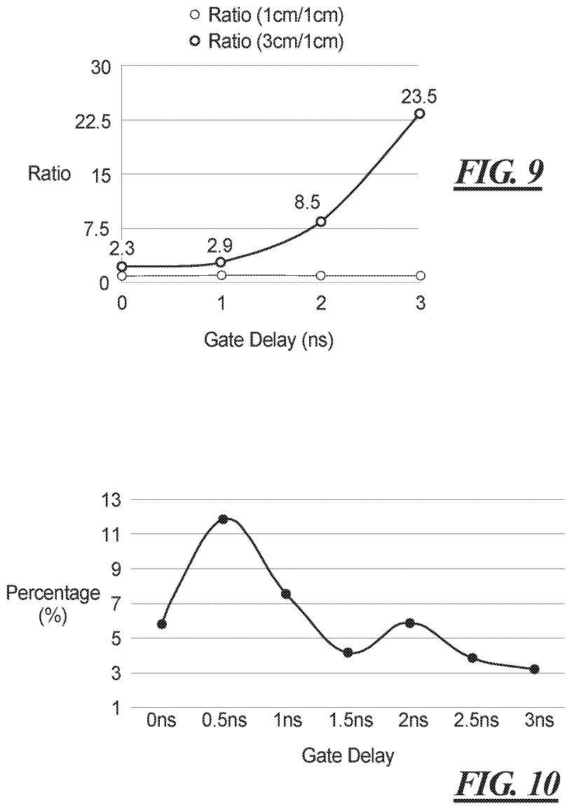

The laser was pulsed on the surface of the blocks and the number of counts was recorded for set of delays between 0 nanoseconds (ns) and 3 ns at 1 ns intervals. At no delay (0 ns), majority of the photons were coming from the surface (see FIG. 9). At a delay of 1 ns, the early incoming photons were blocked and the photons received have a travel time corresponding to a particular depth. To quantify the deeper penetrating photons, we used the ratio of the number of photons coming from the 3 cm block to the number of photons coming back from 1 cm block for each delay as a marker. We observed a 23.5 times improvement in the number of photons coming from greater than 1 cm depth with a gate delay of 3 ns (see also, FIG. 9).

A second set of experiments were performed with the set of liver samples. The system included optical fibers coupled to the laser and the SPAD detector, with the ends of the optical fibers at the working end being spaced approximately 1 mm apart.

The laser was pulsed on the surface of the samples and the number of counts was recorded for set of delays between 0 nanoseconds (ns) and 3 ns at 0.5 ns intervals. For gate delay of 0.5 ns, the number of photons collected from 3 cm block improved significantly as compared to the ones collected from the 1 cm block (FIG. 10). It is believed that this is an indication that the SPAD detector operated with gate delays of 0.5 ns and greater is collecting the photons mostly coming from 1 cm depth and beyond.

In conclusion, although the preceding text sets forth a detailed description of different embodiments of the invention, it should be understood that the legal scope of the invention is defined by the words of the claims set forth at the end of this patent. The detailed description is to be construed as exemplary only and does not describe every possible embodiment of the invention since describing every possible embodiment would be impractical, if not impossible. Numerous alternative embodiments could be implemented, using either current technology or technology developed after the filing date of this patent, which would still fall within the scope of the claims defining the invention.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112(f).

1. A surgical system used to detect tissue and/or determine tissue characteristics within a region proximate to a working end of a surgical instrument, comprising:
at least one light emitter disposed at the working end of the surgical instrument and configured to be activated to emit photons;
at least one light sensor disposed at the working end of the surgical instrument, facing in a common direction as the at least one light emitter, and configured to receive photons emitted from the at least one light emitter and exiting from the region, the at least one light sensor configured to receive photons over a limited time period; and
a controller coupled to the at least one light sensor, the controller configured to operate the at least one light sensor to receive photons over the limited time period after a time delay from activation of the at least one light emitter.

2. The surgical system according to claim 1, wherein the at least one light emitter is a pulsed laser.

3. The surgical system according to claim 2, further comprising a length of single mode optical fiber, the length of optical fiber having a first end optically coupled to the laser and a second end disposed at the working end of the surgical instrument.

4. The surgical system according to claim 1, wherein the at least one light sensor is a time-gated single photon avalanche diode detector.

5. The surgical system according to claim 4, further comprising a length of multimodal optical fiber, the length of optical fiber having a first end optically coupled to the single photon avalanche diode detector and a second end disposed at the working end of the surgical instrument.

6. The surgical system according to claim 1, further comprising a photon counter and a delay circuit, the photon counter coupled to the at least one light sensor and the at least one light emitter coupled to the photon counter via the delay circuit.

7. The surgical system according to claim 1, wherein the controller varies the time delay from activation of the at least one light emitter.

8. The surgical system according to claim 1, wherein the controller is configured to repeatedly cycle activation of the at least one light emitter and operation of the at least one light sensor after the time delay from the activation of the at least one light emitter.

9. The surgical system according to claim 1, wherein the controller is configured to determine a first pulsatile component and a second non-pulsatile component of an output of the at least one light sensor, and to use at least one of the first pulsatile component and the second non-pulsatile component to detect tissue and/or determine tissue characteristics within the region proximate to the working end of the surgical instrument.

10. The surgical system according to claim 9, wherein the controller is configured to adapt an emission intensity of the at least one light emitter according to the second non-pulsatile component.

11. A method of determining detecting tissue and/or determining tissue characteristics within a region proximate to a working end of a surgical instrument, comprising:
emitting photons at the working end of the surgical instrument in the direction of a surface of the region;
sensing photons exiting from the surface at the working end of the surgical instrument over a limited time period delayed from the emitting of the photons;
generating a signal based on the photons sensed at the working end of the surgical instrument; and
detecting tissue and/or determining tissue characteristics within the region proximate to the working end of the surgical instrument based on the signal.

12. The method according to claim 11, wherein the delay of sensing photons from the emitting of the photons is varied.

13. The method according to claim 11, wherein the emitting photons and the sensing photons over a limited time period delayed from the emitting of the photons are repeated cycled.

14. The method according to claim 11, further comprising splitting the signal based on the photons sensed into a first pulsatile component and a second non-pulsatile component, and at least one of the first pulsatile component and the second non-pulsatile component is used in detecting tissue and/or determining tissue characteristics within the region proximate to the working end of the surgical instrument.

15. The method according to claim 11, further comprising splitting the signal based on the photons sensed into a first pulsatile component and a second non-pulsatile component, and using the second non-pulsatile component to adapt an emission intensity.
